(19) United States
(12) Patent Application Publication
(10) Pub. No.: US 2014/0012316 A1
(43) Pub. Date: Jan. 9, 2014

(54) WOUND CLOSURE INVOLVING SILICONE

(71) Applicant: Howard D. Stupak, Westport, CT (US)

(72) Inventor: Howard D. Stupak, Westport, CT (US)

(21) Appl. No.: 14/020,682

(22) Filed: Sep. 6, 2013

Related U.S. Application Data
(63) Continuation-in-part of application No. 13/407,476, filed on Feb. 28, 2012.
Application No. 13/407,476 is a continuation-in-part of application No. 12/313,599, filed on Nov. 21, 2008, now Pat. No. 8,267,962.
(60) Provisional application No. 61/743,535, filed on Sep. 6, 2012.
(60) Provisional application No. 61/005,158, filed on Dec. 3, 2007.
(60) Provisional application No. 61/011,026, filed on Jan. 14, 2008.
(60) Provisional application No. 61/070,646, filed on Mar. 25, 2008.

(51) Int. Cl. A61B 17/064; A61B 17/04

(52) U.S. Cl. CPC A61B 17/0644; A61B 17/0466
USPC 606/219

(57) ABSTRACT

Apparatus and methods of closing wounds using one or more plates comprising silicone which can be either sutured to the opposing edges of a wound or affixed to the opposing edges of the wound using rigid legs which penetrate tissue adjacent to the wound. The plate is brought into contact with the tissue as the wound is closed such that the plate compresses the tissue during healing.

### CROSS-REFERENCE TO RELATED APPLICATIONS

This application claims the benefit of provisional patent Application No. 61/743,535 filed Sep. 6, 2012 and is a continuation-in-part of U.S. patent application Ser. No. 13/407,476, filed Feb. 28, 2012, which is a continuation-in-part of U.S. patent application Ser. No. 12/313,599, filed Nov. 21, 2008, which claims the benefit of provisional patent Application Nos. 61/005,158, filed Dec. 3, 2007, 61/011,026, filed Jan. 14, 2008, and 61/070,646, filed Mar. 25, 2008, all of which are incorporated by reference as if fully set forth herein.

### BACKGROUND OF THE INVENTION

Silicone has been demonstrated to reduce hypertrophic, keloid, and other scars when applied to a wound. Silicone bandages, creams, and pads can be placed over a closed, healing wound in order to reduce scarring. However, bandages, creams, or pads cannot eliminate the need for suturing or stapling in order to close the wound, and therefore they must be used after the wound has been sutured. For that reason, silicone is conventionally used only after sutures have been removed, which is after the wound has already been sealed and after scars have already begun to develop.

Silicone sheeting is a widely used and proven technique in the rehabilitation of hypertrophic, keloid, and other unfavorable scars. For example, U.S. Pat. No. 6,870,074 discloses a thin scar dressing made of silicone that is applied to a healing wound which has been closed through other means. However, this disclosure teaches placement of the silicone sheets after the wound has been closed using conventional wound closure devices and methods.

Conventional wound closure methods include sutures and staples. Suturing stitches tissues together using a needle and attached thread. Surgical staples are typically made of surgical steel. They consist of a spine and legs. The legs are driven into skin on either side of the wound, the spine bridges the wound, and the legs draw the tissue together, bringing the opposing sides of the wound together. Before application, they can be preshaped into an “M” shape, with the spine bent and the legs straight. When they are applied to fasten tissues together, the spine is deformed, which pushes the legs through the skin and into the fascia. To remove a surgical staple, the spine is bent so that the legs are laterally displaced.

These conventional wound closure devices often exacerbate scarring. Staples contact the surface of the tissue beneath the spine, such that the spine compresses the healing tissue in narrow strips across the wound. These narrow bands of compression can create a “train track” scar. Additionally, tension is concentrated where the legs of the staple enter the skin, creating distortion in the healing skin.

Conventional suturing and stapling can lead to a complication known as “pin-cushioning” when wound edges are uneven, or when uneven tensioning of tissues by sutures pushes tissue edges up. Additionally, staples and sutures cannot be repositioned and their tension cannot be adjusted without disturbing the healing tissue.

A skin staple that avoided these drawbacks and enabled the involvement of silicone in wound closure would be advantageous.

### SUMMARY OF THE INVENTION

Disclosed herein is a method of closing a wound having at least two edges, comprising: providing a device comprising a central bridge portion joined to two or more legs, said central bridge portion comprising a semi-rigid plate comprising silicone and having a first surface and a second surface; simultaneously closing said wound and bringing said first surface of said plate into contact with said wound by deforming said device and causing each said leg to penetrate at least one said edge of said wound; and compressing said wound with said first surface of said plate. Also disclosed herein is a method of closing a wound in tissue having at least two edges, comprising; providing a semi rigid plate comprising silicone having a first surface and a second surface; simultaneously removably attaching said device to said at least two edges, bringing said at least two edges into proximity with one another, and bringing said plate into contact with said at least two edges; and compressing said wound with said first surface of said plate.

The foregoing and other objectives, features, and advantages of the invention will be more readily understood upon consideration of the following detailed description of the invention taken in conjunction with the accompanying drawings.

### BRIEF DESCRIPTION OF THE DRAWINGS

FIG. 1 is an enlarged perspective top view of one embodiment of a wound closure device having a spinal structure composed of a silicone plate and attached legs.

FIG. 2 is an enlarged perspective top view a row of wound closure devices used to close a wound.

FIG. 3 is a top perspective view of a skin closure device with legs in a splayed position, pre-insertion.

FIG. 4 is a top perspective view of three skin closure devices of an embodiment of a wound closure device stacked in a pre-insertion configuration.

FIG. 5 is a top perspective view of an embodiment of a wound closure device being used to close a wound.

FIG. 6 is a top perspective view of two wound closure devices of an additional embodiment of the invention stacked in a pre-insertion configuration.

FIG. 7 is a top perspective view of an additional embodiment of a wound closure device.

FIG. 8 is a top perspective view of an additional embodiment of a wound closure device in a pre-insertion configuration.

FIG. 9 is a top perspective view of an additional embodiment of a wound closure device.

FIG. 10 is a top perspective view of an additional embodiment of a wound closure device in pre-insertion configuration.

FIG. 11 is a top perspective view of an additional embodiment of a wound closure device being used to close a wound.

FIG. 12 is a perspective view of an additional embodiment of a wound closure device disclosed herein being used to close a wound.

FIG. 13 is a perspective view of an additional embodiment of a wound closure device disclosed herein closing a wound.

FIG. 14 is a perspective view of a wound that has been closed with one embodiment of a wound closure device disclosed herein.

FIG. 15 is a perspective view of a wound that has been closed with one embodiment of a wound closure device disclosed herein.

### DETAILED DESCRIPTION

The wound closure devices disclosed herein facilitate the immediate application of silicone to a wound by incorporating it into a skin closure device. Various embodiments of the skin closure device disclosed herein offer additional advantages, such as ease of application, prevention of wound desiccation, and mitigation of the scarring caused by conventional wound closure methods.

Clinical results indicate that the use of a plate containing silicone in addition to a suture to close a wound inhibits the progression and development of hypertrophic and keloid scars in preliminary clinical results. (Stupak et al, at the time of filing at press, Ear, Nose and Throat Journal, 2013) As described in U.S. patent application Ser. No. 13/407,476, incorporated herein by reference, the inventor of the devices disclosed herein describes a sutured wound closure device using this immediate application of silicone with wound edge tension redistribution. The benefits of the incorporation of silicone into wound closure can also be obtained if a silicone plate is incorporated into a device having stiff, staple-style appendages which are driven into skin adjacent to the wound and used to close the skin and bring the skin into contact with the silicone plate during healing.

Staples are a conventional wound closure system. A wound can typically be closed with staples faster than with suturing. Staples are typically made of steel or titanium, but can be made partially or completely out of stiff, resilient polymers. As with suturing, however, staples create narrow bands of pressure across a healing wound, resulting in the exacerbation of scarring.

Disclosed herein is a wound closure device 2 that has a central bridge portion 4 which includes a plate 6, which can be joined to a spine 8 or can be used instead of a spine. The plate 6 has a substantial surface area. The distal surface 10 of the plate 6 contacts the surface 12 of skin bearing a wound 14 when the device is in use. The surface area better distributes pressure across the surface of a healing wound instead of concentrating it at the points where legs of a conventional staple enter the skin or in a narrow track across the top of the wound. The plate also has a compressive effect which further inhibits scarring in part by keeping wound edges together and flat. The plate covers the wound, and therefore can prevent desiccation. Finally, the plate preferably comprises silicone which inhibits scarring. The wound closure device may be used for scar prevention in delicate scar revision, facial, cosmetic or flap surgery, and may also aid in closure of difficult high tension wounds.

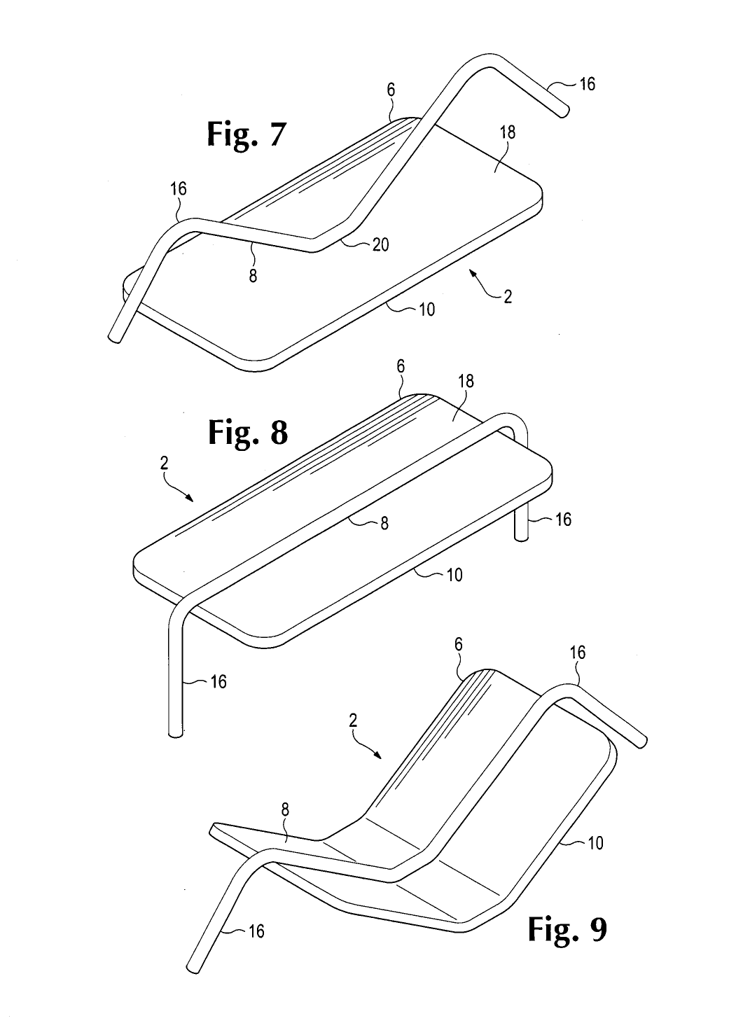

The central bridge portion 4 comprises a plate 6. It may be any size or shape, so long as it has a width that exceeds the width of the legs 16 and provides a relatively large surface area which can contact the surface of the wound. It may be square, or it may be rectangular or oblong. It may be sized to cover a larger wound or portion of a wound with a single device. The plate may be composed of a thin sheet of hard-grade silicone. It may also be made of a thin sheet of more flexible silicone. The silicone may be adhered to or may partially or completely envelope a metal or polymer component such as a spine 8, which lends rigidity, resilience, deformability to the bridge portion. The spine 8 may be a thin metal strip, as shown in FIGS. 8, 9, and 10, or it may have a size or shape which is more commensurate with the size and shape of the plate 6.

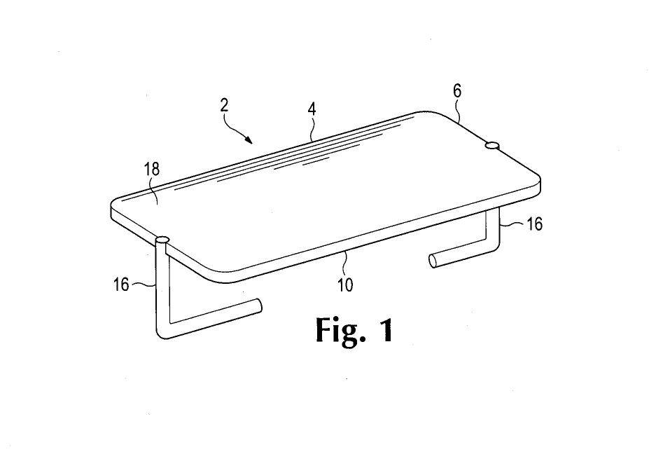

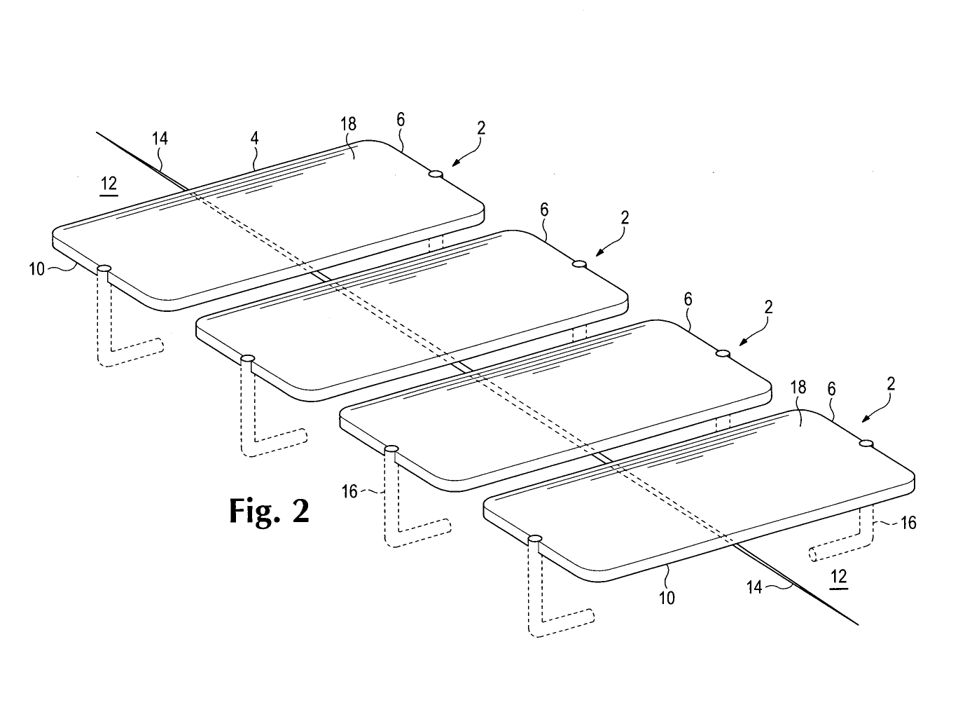

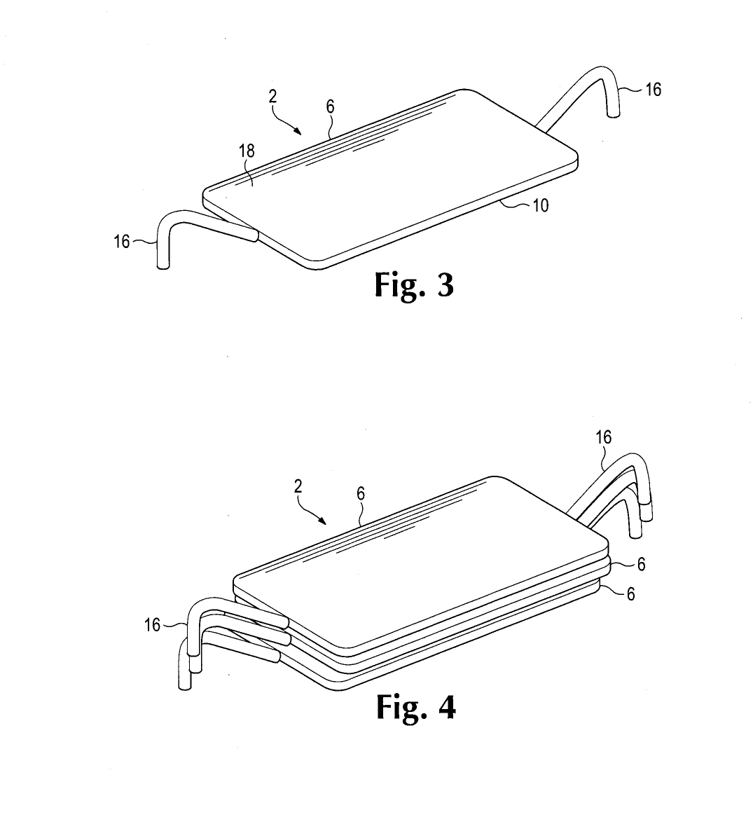

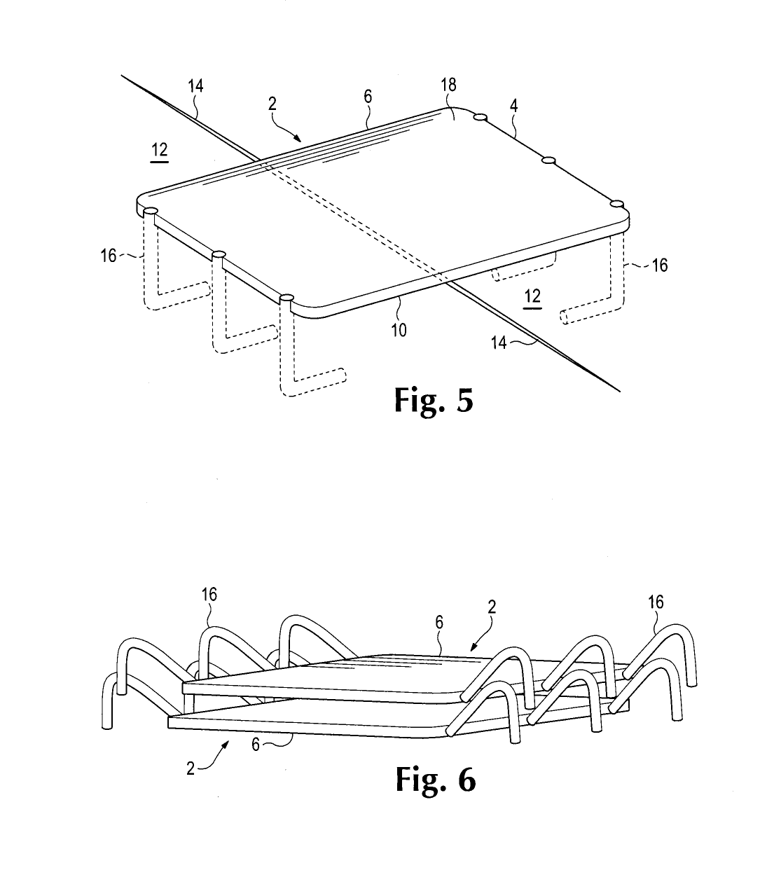

Two or more opposing legs 16 are used to affix the wound closure device to the skin and to draw the edges of a wound together in order to close the wound. The legs 16 should preferably be bent, for example in right angles as shown in FIG. 1, or in a more curved configuration as shown in FIG. 3, but they may also be straight. The device may have two opposing legs or it may employ multiple legs, as shown in FIGS. 5 and 6. Especially if the device has multiple legs, individual legs can be adjusted during healing in order to redistribute tension and further manage healing in a manner which minimizes scarring. The legs may be joined to or integral with a metal spine, or they may be joined to the bridge portion or central plate.

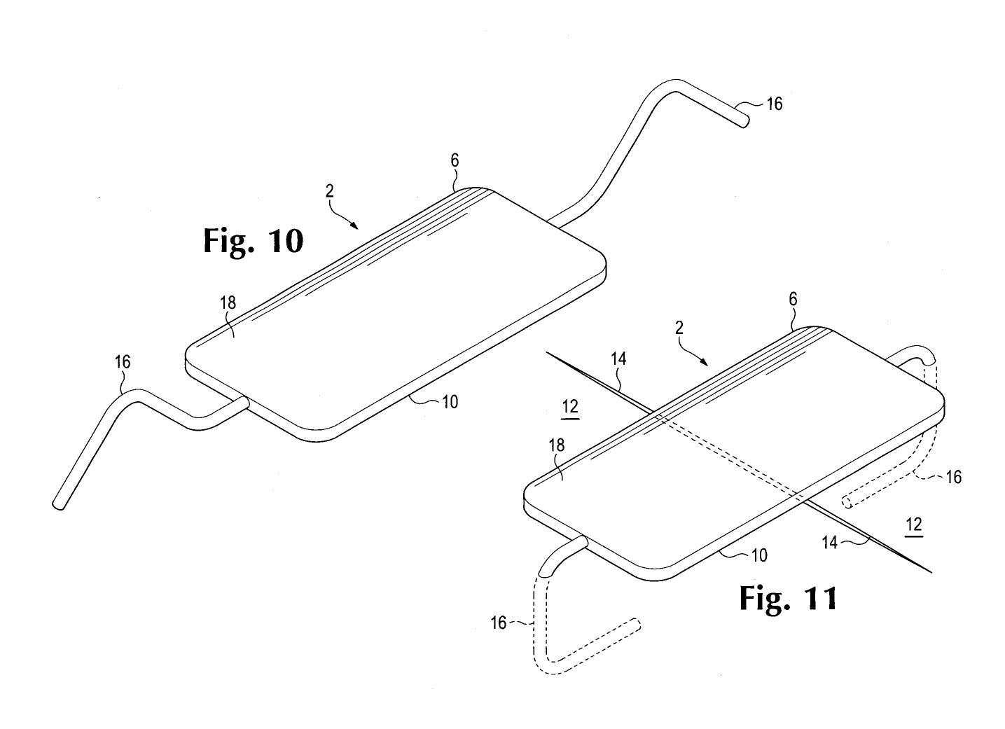

Skin closure devices may be positioned before use with their legs 16 splayed in either straight or partially bent configurations so that they are generally nearly parallel to the bridge portion 4, as shown in FIGS. 4, 6, 8, and 10. When the devices are used, a specialized apparatus is used to force the legs into a flexed position so that they penetrate the skin at an appropriate angle so that the legs simultaneously pull the skin together, close the wound, and bring the plate into contact with wounded skin. The legs may be bent at approximately 90 degree angles when splayed in their pre-insertion position. The legs may then be bent 90 degrees further at the joint where they interface with the spine or plate in order to insert them into skin, bringing the opposing edges of the wound together, as shown in FIGS. 10 and 11. The device maintains this post-insertion shape for the duration of healing. It is removed by again deforming it so that the legs can be retracted out of the skin.

As shown in FIGS. 3, 4, and 6, the plate 6 and bridge portion 4 may remain straight and the legs 16 may be individually flexed up into a pre-insertion position and downward into an insertion position for use as shown in FIG. 5. The legs may be individually bent into insertion position whether they are joined to a spine or not, but, as shown in FIGS. 8, 9, and 10, the legs 16 may be integral with or joined to a spine 8, and the spine may be bent in order to cause the legs to assume pre-insertion positions. If the plate is rigid, as shown in FIG. 8, the spine 8 may be affixed to the proximal surface 18 of the plate 6 at an approximately central point 20 so that when the spine is bent into a pre-insertion position, the legs form an M shape and angle away from the proximal surface 18 of the plate 6. As shown in FIG. 10, if the plate is flexible, a substantial portion of the spine 8 may be affixed to the proximal surface 18 of the plate 6 and when the spine 8 is bent into a pre-insertion configuration, the plate will be bent as well.

The procedure used to place and operate this device has not been described in the previous medical literature or prior art. The device may be used in a soft-tissue incision, wound, or to close a tissue flap to intact tissue. This may be used with endoscopic guidance for any tissue or body cavity, as no knot tying is required.

The procedure itself begins with appropriate anesthesia for the patient. Once adequate subcutaneous or submucosal stitches are placed if necessary, the device may be used. The device should be used instead of mucosal or cutaneous sutures, in areas where there may be a high risk for scarring or increased risk of vascular compromise to tissue, such as the corner of a rotational flap where maximal tension occurs, or where previous scars have already been excised. The practitioner approximates the edges of the wound, and then uses an apparatus to place one or more wound closure devices proximate to one another until the wound is closed. The apparatus used to place the device can preferably be used to modify the position of any one leg in order to change the tension distribution to accommodate changes in the tissue due to edema or healing.

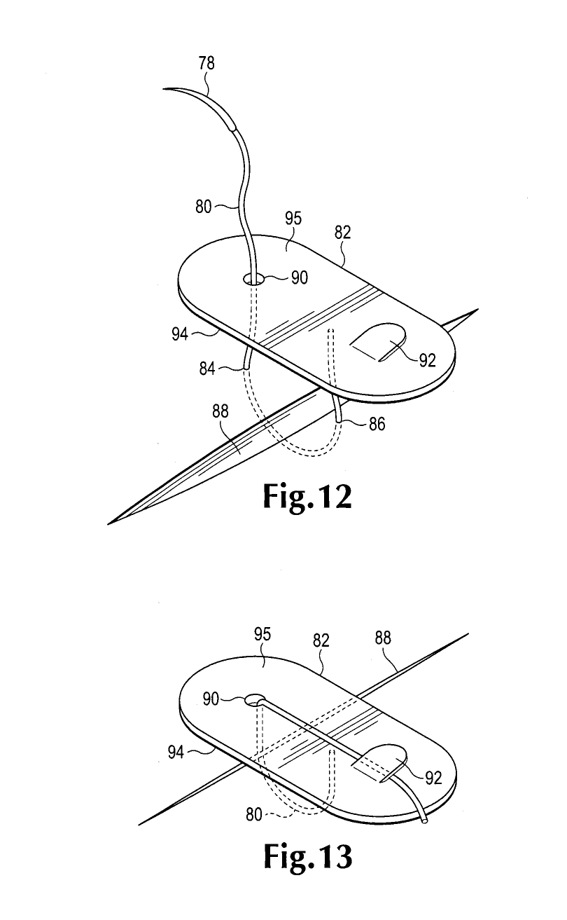

Referring now to FIGS. 12-15, in an additional embodiment, a wound closure device comprises a suture 80 with a surgical needle 78 on its free end, harnessed to a plate 82. The plate 82 may be composed of a silicone sheet or molded plate. Its size and shape may vary depending on the shape and size of the wound and its anatomic site. The plate should have sufficient rigidity to exert pressure on the healing wound, thus limiting scar progression through compression. The plate may include one or more portals 90 such as eyeholes or notches which direct the suture from a first or wound-facing side of the plate 94 to the second or top side of the plate 96. It may also include a mechanism for fastening the suture to the plate, such as a cleat 92 or other fastener for suture securing. In an alternate embodiment, shown in FIG. 15, the plate may have two or more portals 90, and no attached suture. In this embodiment, the suture can be knotted on top of the device.

The device is used to proximate the edges of a wound to close the wound, and can then compress the surface of the closed wound, with tension distributed along the full area of the plate instead of the very narrow width of the suture thread as with conventional suturing. Because of the dynamic attachment of the suture to the plate via the eyehole or notch, pulling from the tissue will result in additional compression from the plate.

In addition, because of the portal fastener components, the suture may be secured without tying a knot, and can have its tension adjusted during the healing process in order to accommodate changes in the tissue caused by edema or healing. Because no suture tying is required, the device can be used for both external and endoscopic procedures where there is limited access to the surgeon for tying knots. Because there is less risk of train-track scarring, the suture plate may be left in place longer, allowing more silicone exposure to the wound, more immobilization, and more support during healing, as well as the prevention of moisture loss from the environment

The procedure itself begins with appropriate anesthesia for the patient. Once adequate subcutaneous or submucosal stitches are placed if necessary, the device may be used. The device should be used instead of mucosal or cutaneous sutures, in areas where there may be a high risk for scarring or increased risk of vascular compromise to tissue, such as the corner of a rotational flap where maximal tension occurs, or where previous scars have already been excised.

The needle 78 and suture 80 harnessed to the plate 80 is introduced into the tissue of one edge 84 of the wound 88, and subsequently passed through an additional edge 86 of the wound 88 as in normal mucosal or dermal skin. Care must be taken to pass through a significant amount of the substance of the subdermal or submucosal tissue to permit adequate eversion and closure.

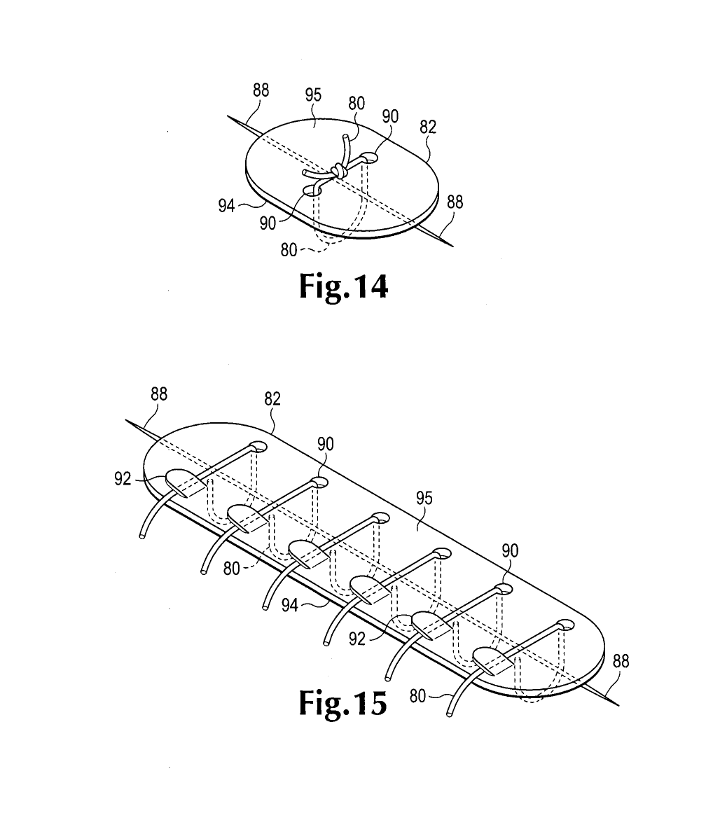

Once passed through, the needle 78 and suture 80 are pulled in a direction away from the tissue. Due to a “pulley” effect, the plate is subsequently urged toward the tissue and into position proximal to the wound 88 at the same time that the edges of the wound 84, 86 are proximated. Before the plate contacts the tissue, the needle and suture are fed through the portal 90, the needle and suture are pulled again, until the plate is maneuvered into position flush with the tissue plane, as shown in FIG. 14, the closed wound 88 at its midpoint (as a flag would be raised on a flagpole).

The wound closes just as the plate becomes flush with the tissue. The desired level of tension is kept on the needle end of the suture, and it is fed into the fastener 92 or tied to secure its position and tension. For additional security, a second fastener, perhaps a v-shaped cut-out in the plate, may be used as well. Optional features are also possible on the plate. A single plate may have multiple sutures and their respective portals or cleats in parallel, as shown in FIG. 16. This would allow one plate to cover a longer incision. The plate may be flat, curved, or oblong shape, depending on the tissue needing repair.

Whether attached to wounded skin with sutures, legs, or other means, the wound closure devices described herein provide compression to the wound edges, maintain the tissues in an appropriate position for healing, and may additionally help protect the wound and help keep the wound moist and clean. Additionally, the use of silicone in contact with the healing tissue immediately upon wound closure further inhibits hypertrophic and keloid scarring. The wound closure devices provide a secured, occlusive dressing that is both rapidly applied and can be partially or completely transparent for visualization of the wound.

The terms and expressions which have been used in this specification are intended to describe the invention, not limit it. The scope of the invention is defined and limited only by the following claims.

What is claimed is:

1. A method of closing a wound having at least two edges, comprising:
a. providing a device comprising a central bridge portion joined to two or more legs, said central bridge portion comprising a plate comprising silicone and having a first surface and a second surface;
b. simultaneously closing said wound and bringing said first surface of said plate into contact with said wound by deforming said device and causing each said leg to penetrate at least one said edge of said wound; and
c. compressing said wound with said first surface of said plate.

2. The method of claim 1 wherein said device has two legs.

3. The method of claim 1 wherein said device has at least four legs.

4. The method of claim 2 wherein said central bridge portion further comprises a spine which is in contact with said plate, and said legs are integral with said spine.

5. The method of claim 3 wherein said device further comprises at least two spines and two said legs are associated with each said spine.

6. A method of closing a wound in tissue having at least two edges, comprising;
a. providing a plate comprising silicone having a first surface and a second surface;
b. simultaneously removably attaching said device to said at least two edges, bringing said at least two edges into proximity with one another, and bringing said plate into contact with said at least two edges; and
c. compressing said wound with said first surface of said plate.

7. The method of claim 6 wherein said device has two legs joined to said central bridge portion, each said leg adapted to penetrate at least one said edge of said wound.

8. The method of claim 7 wherein said device is deformed into a penetration position as it is removably attached to said at least two wound edges.

9. The method of claim 8 wherein said central bridge portion further comprises a spine which is in contact with said plate, and said legs are integral with said spine.

10. The method of claim 8 wherein said legs are deformed.
